(19) United States
(12) Patent Application Publication
(10) Pub. No.: US 2020/0338018 A1
(43) Pub. Date: Oct. 29, 2020

(54) IMPLANTABLE DEVICES WITH INTEGRATED CBD DELIVERY MECHANISMS

(71) Applicant: Adam M. Rotunda, Irvine, CA (US)

(72) Inventor: Adam M. Rotunda, Irvine, CA (US)

(21) Appl. No.: 16/855,818

(22) Filed: Apr. 22, 2020

Related U.S. Application Data
(60) Provisional application No. 62/837,621, filed on Apr. 23, 2019.

(51) Int. Cl. A61K 31/05; A61B 17/70; A61B 17/80; A61B 17/84; A61B 17/86; A61F 2/24; A61M 1/10; A61F 2/82; A61N 1/362; A61N 1/39; A61F 6/14; A61F 2/00; A61B 17/064; A61B 17/06; A61M 27/00; A61M 37/00; A61F 2/16; A61M 5/142; A61C 8/00; A61M 39/02; A61K 9/00

(52) U.S. Cl. CPC A61K 31/05; A61B 2017/00893; A61B 17/80; A61B 17/846; A61B 17/86; A61F 2/24; A61M 1/10; A61F 2/82; A61N 1/362; A61N 1/3956; A61F 6/14; A61F 2/0059; A61B 17/064; A61B 17/06166; A61M 27/00; A61M 37/0015; A61F 2/16; A61M 5/14276; A61C 8/0013; A61M 39/0208; A61K 9/0024; A61B 17/7002

(57) ABSTRACT

Cannabidiol (CBD) is one of the most abundant and physiologically active phytocannabinoids in Cannabis plants. The chemical has well-known mechanisms that have been the potential to treat and prevent numerous inflammatory, ischemic, psychological, immunologic, infectious, oxidative and neurogenic-related diseases. The incorporation of CBD on and/or within medical devices and implants is a novel pharmacologic approach to alleviate, prevent and cure local as well as systemic disease, and promote healing and recovery.

### CROSS-REFERENCE TO RELATED APPLICATIONS

This application claims priority to U.S. Application Ser. No. 62/837,621 filed Apr. 23, 2019, the teachings of which are expressly incorporated herein by reference.

### STATEMENT RE: FEDERALLY SPONSORED RESEARCH/DEVELOPMENT

Not Applicable

### BACKGROUND

### 1. Technical Field

The present disclosure relates generally to the field of therapeutic uses of cannabinoids. More particularly, the present disclosure relates to the integration of cannabidoil delivery mechanisms into implantable devices.

### 2. Related Art

Cannabinoids are a diverse set of chemical compounds that bind to special receptors in the human body that make up the endocannabinoid system. It has been known for some time now that the human body generates endogenous substances that bind to cannabinoid receptors, such as anandamide and 2-arachidonoylglyceral (2-AG). Several endocannabinoids also bind to other receptors, such as the GPR55 receptor and vanilloid receptors. Currently, it is thought that more than 200 endocannabinoids and related substances exist which are produced by the human body and either bind to the cannabinoid receptors or otherwise complement the function of endocannabinoids.

The main exogenous source of cannabinoids are phytocannabinoids, which are found primarily in the Cannabis plant, as well as in certain other plants, including E. purpurea, E. angustifolia, A. olaracea, Helicrysum, and R. marginata. In the Cannabis plant, phytocannabinoids and terpenes are manufactured in resin glands (trichomes) present on the flowers and the main fan leaves of late-stage Cannabis plants.

Over 100 phytocannabinoids have been identified as deriving from the Cannabis plant. The most studied phytocannabinoids are tetrahydrocannabinol (THC) and cannabidoil (CBD).

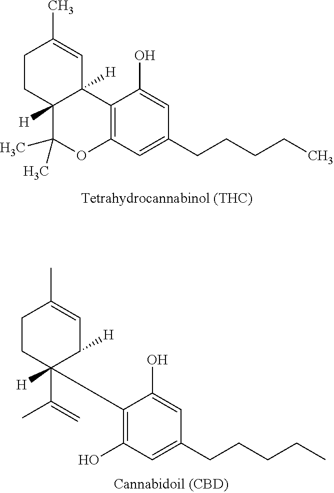

THC is generally considered to be the primary psychoactive phytocannabinoid derived from the Cannabis plant. In the Cannabis plant, THC is produced via the decarboxylation of its precursor tetrahydrocannabinolic acid (THCA), with the decarboxylation conversion process being accelerated via the drying of the Cannabis plant, and rapidly accelerated when THCA is heated or burned.

CBD is generally considered to be a non-psychoactive phytocannabinoid derived from the Cannabis plant. Similar to THC, CBD is produced via decarboxylation from its precursor cannabidiolic acid (CBDA) in a similar fashion. However, CBD is considered to have little binding affinity for either of the two confirmed cannabinoid receptors, CB₁ and CB₂. Rather, CBD modulates several non-cannabinoid receptors and ion channels, as well as acting through various receptor-independent pathways, such as by delaying the reuptake of endogenous neurotransmitters (such as anandamide and adenosine) and by enhancing or inhibiting the binding action of certain G-protein coupled receptors.

One interaction of CBD that has been discovered is that it serves as a partial agonist of the 5-HT_{1A} (hydroxytryptamine) serotonin receptor, which is a G protein-coupled receptor that mediates inhibitory neurotransmission, resulting in relief of anxiety and depression in humans. Multiple anxiolytic and antidepressant partial or full 5-HT_{1A} receptor agonists, such as buspirone and tandospirone, are presently in common medical use.

Another interaction of CBD is with the transient receptor potential cation channel subfamily V member 1 (TrpV1) receptor, also known as the capsaicin receptor and the vanilloid receptor 1. The TrpV1 receptor mediates pain and temperature perception. Currently, some products are in clinical use which rely on TrpV1 receptor agonists, such as capsaicin and resiniferatoxin, for the alleviation of localized pain via prolonged application resulting in long term desensitization of those receptors. For example, 8% capsaicin patches which rely on this interaction have recently entered into clinical use, with novel preparations containing higher amounts of capsaicin under clinical trials. Thus, CBD may also provide alleviation of medical conditions via these interactions as well.

Recent studies have also indicated that CBD may function as an antagonist for the G protein-coupled receptor 55 (GPR55). GPR55 is widely expressed in the brain, especially in the cerebellum, but it is currently considered to be an “orphan receptor,” as its physiological function remains unclear and there remains certain uncertainty as to whether it belongs to a larger family of receptors, such as the cannabinoid receptors, and should be considered to be the “CB₃” receptor. Some research has indicated that GPR55 receptor activation increases the level of intracellular calcium and inhibits M current. There also is a growing body of evidence that GPR55 may play a role in controlling cell proliferation, and thus may be implicated as a biomarker or target in certain cancer therapies. According to some sources, THC operates as an agonist for the GPR55 receptor, causing its activation, while CBD is one of its few known antagonists, preventing its activation. Accordingly, administration of CBD therapeutics may have a substantial effect with regard to the conditions or other therapeutics activating or affected by activation of the GPR55 receptor.

These are only some of the known or suspected biological interactions of CBD. Thus, CBD may be seen to have many potential uses as a as a therapeutic agent for numerous pathological conditions. Known pharmacologic effects of CBD include anti-oxidation, anti-inflammation, anti-bacterial, analgesia (anti-pain), neuroprotection, immunomodulation, anti-psychotic, anti-fibrosis, anti-coagulation, anti-nausea, anti-fatigue and muscle tension, anti-depression, and anti-convulsant activity. These effects have current or potential applications in: Alzheimer's disease, Parkinson's disease, multiple sclerosis, epilepsy, Huntington's disease, pain, hypoxia-ischemia injuries (i.e., cardiovascular and nerve disease), cancer, anxiety, depression, hypertension, nausea, inflammatory diseases (i.e., bowel disease, arthritis, cardiovascular disease, autoimmune diseases), and complications from diabetes.

An implant is a medical device manufactured to replace a missing biological structure, support a damaged biological structure, or enhance an existing biological structure. They are synthetic devices (in contrast to a transplants). The surface of implants that contact the body may be made of a biomedical material such as titanium, silicone, or apatite depending on what is the most functional. In some cases, implants contain electronics (e.g., artificial pacemaker and cochlear implants). Some implants are bioactive, such as subcutaneous drug delivery devices in the form of implantable pills or drug-eluting stents.

Numerous medical and surgical specialties have utilized implants to augment, enhance, or replace the structure and function of the target organ system. Examples include: orthopedic implants (rods, screws, pins, plates, etc.) to treat fractures, arthritics, scoliosis stenosis and chronic pain; electrical implants (electrodes) to treat rheumatoid arthritis and neuropathic pain; cardiovascular implants (valves, stents, pacemakers, defibrillators, artificial hearts) to treat the heart and vasculature system; contraceptive implants (intrauterine devices) to prevent unintended pregnancy; cosmetic implants (dermal fillers, breast implants, and prosthesis) to restore cosmesis and counteract disfigurement; and other types of implants (dental implants, gastrointestinal implants, surgical meshes, penile).

Some medical devices, which are instruments, in vitro reagents, or other similar related materials that do not depend upon being metabolized for their primary intended purpose, are used to affect the structure or function of the body. Specifically, surgical devices like sutures or staples are medical devices that are used to hold body tissues together after an injury or surgery. A number of different shapes, sizes, and thread materials have been developed over their history of use. Other surgical devices, such as tubing, drains, surgical gloves, instruments, and needles are used commonly in the medical setting. As such their sterility and anti-bacterial properties are requisite in certain clinical and surgical environments to minimize infection risk and microorganism disease.

At times, medical devices and medical implants are impregnated with chemicals and drugs to treat or prevent disease, minimize adverse events, surgical complications and disease progression. Examples include drug eluting stents used in coronary or peripheral artery disease that slowly releases a drug to block cell proliferation, prevent fibrosis and block clots that could otherwise block the stented artery (restenosis). Other examples of drug releasing devices are contraceptives (intra-uterine devices (IUDs) that release steroids); drug delivery within cataract lenses; orthopedic implants (coated with nanostructured, biodegradable polymer films that release drug into the local environment to prevent bacterial (infectious) biofilms or reduce inflammation; dental implants with reservoirs for slow releasing drugs (to reduce bacterial biofilms); bactericidal surgical suture coatings; and implantable chips (for drug delivery, for the treatment of pain, contraception, osteoporosis, and diabetes, for example).

Because investigation into the therapeutic potential of CBD is ongoing, there is likewise a need in the art for novel ways to synergistically integrate the therapeutic capabilities of CBD with the therapeutic effects of implantable medical devices, especially when CBD is delivered at the site of implantation, rather than systemically delivered.

### BRIEF SUMMARY

To solve these and other problems, according to various exemplary embodiments, an augmented medical implant is contemplated as comprising an implantable medical device configured to perform at least a first medical function ensuing from the implantation of the implantable medical device at a site of implantation, and a drug release mechanism in physical association with the implantable medical device, with the drug release mechanism being configured to deliver cannabidiol at the site of implantation, and with the delivery of cannabidiol at the site of implantation being configured to augment the performance of the first medical function by the implantable medical device.

According to various of these exemplary embodiments, the implantable medical device may be selected from one or more of: an orthopedic implant, an electrical implant, a cardiovascular implant, a contraceptive implant, a cosmetic implant, a dental implant, a gastrointestinal implant, and a surgical implant.

Where the implantable medical device comprises an orthopedic implant, the implantable medical device may be, for example, selected from one or more of: rods, screws, pins, plates, or combinations thereof.

Where the implantable medical device comprises a cardiovascular implant, the implantable medical device may be, for example, selected from one or more of: a valve, a stent, a pacemaker, a defibrillator, an artificial heart.

Where the implantable medical device comprises a contraceptive implant, the implantable medical device may be, for example, selected from one or more of: an intrauterine device, a hormone-releasing device, an injectable depot.

Where the implantable medical device comprises a cosmetic implant, the implantable medical device may be, for example, selected from one or more of: an injectable filler, a prosthesis, a graft, or combinations thereof.

Where the implantable medical device comprises a surgical implant, the implantable medical device may be, for example, selected from one or more of: a tubing, a drain, an instrument, a needle, a stent, a suture, a staple, a thread, or combinations thereof.

It is also contemplated that, according to various of the exemplary embodiments, the drug release mechanism configured to deliver cannabidiol at the site of implantation may comprise one or more of: a coating, a depot, a reservoir, a microfluidic device, and combinations thereof.

Where the drug release mechanism configured to deliver cannabidiol at the site of implantation comprises a coating, the coating may be, for example, selected from: a polymer coating, a cement coating, a biocompatible coating, a non-biologic coating, or combinations thereof.

Where the drug release mechanism configured to deliver cannabidiol at the site of implantation comprises a microfluidic device, the microfluidic device may be, for example, selected from: an actively driven reservoir, a passive micropump, an electrostatic micropump, an active micropump.

It is also contemplated that the drug release mechanism configured to deliver cannabidiol at the site of implantation may comprise an embedded fluid port to permit the drug release mechanism to refilled with cannabidoil.

It is additionally contemplated that the drug release mechanism configured to deliver cannabidiol at the site of implantation may comprise a reservoir associated with a controller, wherein the controller is operative to actuate release of cannabidoil from the reservoir. The controller may be configured to be operative to actuate release of cannabidiol from the reservoir based upon a number of different criteria, including, for example and without limitation, based upon a predetermined timing, based upon receipt of a release command, or based upon detection of a physiological condition. The controller is contemplated in certain embodiments as comprising a microchip.

Where the drug release mechanism configured to deliver cannabidiol at the site of implantation comprises a depot, the depot may be, for example, selected from: a polymer formulation, a liposomal formulation, a micro-encapsulation formulation, an ion-exchange resin formulation, or combinations thereof.

According to one particular exemplary embodiment, the drug release mechanism configured to deliver cannabidiol at the site of implantation comprises an adherent coating configured to elute cannabidoil, and the physical association with the implantable medical device comprises the adherent coating being adhered to at least a portion of a surface of the implantable medical device. It is contemplated that according to this embodiment, the implantable medical device may comprise one or more of a stent, a tubing, a drain, an intra-ocular lens, a microneedle, a microcapsule, a microresevoir, a micropump, or combinations thereof.

### DETAILED DESCRIPTION

According to various embodiments of the present disclosure, medical implant systems are contemplated in which an implantable medical device configured to perform a first medical function is augmented by the physical association therewith of a drug release mechanism configured to delivery cannabidoil (CBD) at the site of implantation, thereby augmenting the performance of the first medical function. As described above, CBD delivery at the site of implantation may be beneficial in treating or aiding in the treatment of a large number of conditions, through administration via a variety of biochemical pathways or combinations of biochemical pathways. Thus, it may be seen that the therapeutic application of CBD for augmentation of the performance of the first medical function of the medical implant may be customized in a variety of ways particular to, among other things, the type of implant, the medical function of the implant desired to be augmented, the condition or conditions being treated by the implantation of the medical implant, and the particular needs of the recipient of the medical implant, which is primarily contemplated to be a human patient but may also be other organism which may benefit from implantation of the presently contemplated medical implant systems.

The following disclosure exemplifies a number of ways in which such contemplated medical implant systems containing a drug release mechanism for the delivery of CBD at the site of implantation may be customized, but it is to be understood that the ways in which such customization may occur is essentially infinite, and as such the foregoing discussed contemplated medical implant systems are to be understood as exemplary and illustrative, and that the scope of the presently contemplated disclosure is not to be merely limited to the particular embodiments discussed herein, but rather as including the full scope of all varieties, combinations, and potential combinations of such customized medical implants.

According to one exemplary embodiment of an augmented medical implant having an implantable medical device with a drug release mechanism being in physical association with the implantable medical device and being configured to deliver cannabidoil at the site of implantation comprises a polymer coating covering or otherwise adhered to at least a portion of an orthopedic implant, with the polymer coating containing CBD. Following implantation, the CBD may dissociate from or otherwise migrate from the polymer coating and to the site of implantation, which may occur via a number of processes, depending on the configuration of the polymer coating. For example, it may be seen that a polymer coating may be configured to break down, dissolve, be bioadsorbed, or otherwise be degraded, consequently causing release of the CBD over time from such degradation. The polymer coating may also not be configured to degrade, but rather to remain in place in association with the orthopedic implant, with the CBD eluting from the polymer coating without degradation, such as in other known processes of releasing confined molecules from within coatings.

It may be seen that in the case that the drug release mechanism is a coating, it may not necessarily be required to be polymer coating, but may be any type of coating or combinations of types of coatings known to be able to confine and subsequently allow release of small molecules such as CBD. For example, such coatings may be, in addition to polymer coatings, cement coatings, biocompatible coatings, non-biologic coatings, or combinations thereof.

In other embodiments, the drug release mechanism may comprise a reservoir, which may be a physical reservoir, with the CBD being contained by itself or in solution with or otherwise in association with other compounds. The reservoir may be, for example, any type of known or future developed reservoir-based drug delivery system. For example, intravitreal implants for implantation in the vitreous of the eye frequently contain drug reservoirs, which may serve to allow long-term sustained release of small molecules such as corticosteroids for periods of years. Some types of reservoirs may deliver their supply passively over time, while in other cases, reservoirs may be used in conjunction with microelectromechanical systems, such as microfluidic systems, in order to enable delivery from the reservoir in other ways (an actively driven reservoir). It may be seen that such reservoirs, and other known and future developed reservoir systems, may be adapted to or suitable for the delivery of CBD. It may also be seen that reservoirs may be provided with an embedded fluid port to permit the reservoir to be refilled. Such reservoirs, rather than the drug delivery mechanism for delivery of CBD itself, may instead be the implantable medical device for treatment of the first medical condition, with the drug delivery mechanism being associated with the reservoir, such as in a coating on the exterior or interior surface of the reservoir which may elute CBD similar to that previously described. It may thus be seen that the reservoir may itself deliver one medicament via the action of the reservoir itself, while CBD is also delivered via elution from the coating. Further, in situations where the coating may be on the interior surface of the reservoir, the delivery action from the reservoir may deliver both the medicament contained within the reservoir as well as CBD that has eluted from the coating and into the reservoir.

Further, it may also be seen that other microfluidic devices in addition to an active or passive reservoir may be utilized as components within the present disclosures. For example, CBD may be contained within a coating applied to, without limitation, an actively driven reservoir, a passive micropump, an electrostatic micropump, or an active micropump, or may otherwise be included within those components in a fashion configured to allow CBD to elute from the component following implantation. Further, it may be seen that electrical components, which may or may not overlap with microfluidics devices may be utilized as well as either the implantable medical device or to form the drug release mechanism. One significant type of implant which is contemplated as being used in association with a CBD eluting drug release mechanism are orthopedic implants, including but not limited to rods, screws, pins, plates, or combinations thereof. Furthermore, surgical implants such as tubings, drains, instruments, needles, stents, sutures, staples, threads, or combinations thereof may also be used in association with a CBD eluting drug release mechanism as described herein.

It may be seen that in such cases where portions of or the entirety of an implant of any type (including but not limited to microfluidic devices) are configured to be bioadsorbed or to be otherwise excreted from the body, CBD may be bound up within the implant and be released at the site of implantation as a consequence of such bioabsorption or excretion. It may also be seen that via modification of the way in which CBD is bound or otherwise contained, the parameters of its release may be affected, as in a passive drug release mechanism such as a depot injection where CBD may be formulated as a component of a polymer formulation, a micro-encapsulation formulation, an ion-exchange resin formulation, a liposomal formulation, or combinations thereof.

It may also be seen that according to other exemplary embodiments, the implantable medical device may be other forms of implants, such as dental implants, cosmetic implants, gastrointestinal implants, cardiovascular implants, or contraceptive implants. For example, a dental implant may include a reservoir or depot which may directly deliver CBD to the intramedullary space in the jaw. It is additionally contemplated that because many contraceptive implants such as hormone-releasing devices are configured to release small molecules, and can be configured to also release CBD via the same or a different mechanism. In the alternative, a non-hormonal contraceptive implant such as an intrauterine device which operates via occlusion may include physically associated therewith any of the drug delivery systems for delivering CBD described herein. Cardiovascular implants such as pacemakers or artificial hearts may be well-suited for association with a CBD releasing drug-release mechanism. Implants such as depot injections may also be seen to be well suited for association with a CBD drug-release mechanism. In particular, implantable devices with numerous applications such as stents may be very well suited for use in an augmented medical implant as presently disclosed, due to their ability for implantation at numerous locations within a patient, which may permit more general use of such devices across specialties, without necessarily requiring narrow tailoring of the CBD-delivery parameters.

Furthermore, it is contemplated that the CBD drug release mechanism may include aspects for active or passive control of delivery beyond mere time-release as a function of chemical properties of the formulation. For example, it may be seen that the drug release mechanism may be controlled by a microchip to release CBD as a function of a number of possible parameters, such as detecting a physiological condition or the receipt of a release command. For example, a system is contemplated in which a patient or doctor may, via transmission of a signal (electrical, radio, light, or otherwise), trigger the drug release mechanism to release CBD, or in which the system may detect a condition such as elevated blood pressure or ocular pressure and trigger release of CBD.

It may be seen that the delivery of CBD according to the presently described systems, at the site of implantation, may aid in synergistically augmenting the performance of at least one of the medical functions of the implantable medical device. For example, in the case of an implant which serves an analgesic or pain-relieving function, the delivery of CBD at the site of the implant may synergistically aid in the analgesic function. Likewise, for implants for treating hypoxia-ischemia injury, CBD delivery at the site of the implant may synergistically aid in the treatment of hypoxia-ischemia injury. It is not critical that the CBD delivery at the site of the implant aids in the treatment of each and every one of the medical functions of any given implants, as many implants are recognized as delivering treatment in a number of aspects. For example, in the case of a prosthetic or cosmetic implant, such as one for correcting an injury or deformity or changing a patient's physical appearance, the synergy flowing from the inclusion of the CBD drug delivery device may be the result of CBD's anti-anxiety effects in combination with the prosthetic or correcting implant relieving existing anxiety in the patient caused from the presence of the injury, deformity, or other undesired aspect of the patient's physical appearance. In this sense, it may be seen that the inclusion of the CBD drug release mechanism provides substantial synergy in the medical aspect of the treatment of anxiety, without necessarily being required to aid in the treatment of other roles which may be played by the implant (i.e. the physical correction of a deformity or the replacement of a missing body part alone).

The above description is given by way of example, and not limitation. Given the above disclosure, one skilled in the art could devise variations that are within the scope and spirit of the invention disclosed herein, including various ways of physically associating the CBD drug release mechanism with the implant, or of configuring the CBD drug release mechanism to release CBD. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An augmented medical implant, the augmented medical implant comprising:
An implantable medical device configured to perform at least a first medical function ensuing from implantation of the implantable medical device at a site of implantation; and
a drug release mechanism in physical association with the implantable medical device, the drug release mechanism being configured to deliver cannabidiol at the site of implantation;
wherein the delivery of cannabidiol at the site of implantation is configured to augment the performance of the first medical function by the implantable medical device.

2. The augmented medical implant of claim 1, wherein the implantable medical device is selected from one or more of: an orthopedic implant, an electrical implant, a cardiovascular implant, a contraceptive implant, a cosmetic implant, a dental implant, a gastrointestinal implant, and a surgical implant.

3. The augmented medical implant of claim 2, wherein the implantable medical device comprises an orthopedic implant selected from one or more of: rods, screws, pins, plates, or combinations thereof.

4. The augmented medical implant of claim 2, wherein the implantable medical device comprises an orthopedic implant and the first medical function comprises treatment of at least one of: a fracture, an arthritic condition, a dysmorphism, chronic pain, or combinations thereof.

5. The augmented medical implant of claim 2, wherein the implantable medical device comprises a cardiovascular implant selected from one or more of: a valve, a stent, a pacemaker, a defibrillator, an artificial heart.

6. The augmented medical implant of claim 2, wherein the implantable medical device comprises a contraceptive implant selected from one or more of: an intrauterine device, a hormone-releasing device, an injectable depot.

7. The augmented medical implant of claim 2, wherein the implantable medical device comprises a cosmetic implant selected from one or more of: an injectable filler, a prosthesis, a graft, or combinations thereof.

8. The augmented medical implant of claim 2, wherein the implantable medical device comprises a surgical implant selected from one or more of: a tubing, a drain, an instrument, a needle, a stent, a suture, a staple, a thread, or combinations thereof.

9. The augmented medical implant of claim 1, wherein the drug release mechanism configured to deliver cannabidiol at the site of implantation comprises one or more of: a coating, a depot, a reservoir, a microfluidic device, or combinations thereof.

10. The augmented medical implant of claim 9, wherein the drug release mechanism configured to deliver cannabidiol at the site of implantation comprises a coating selected from: a polymer coating, a cement coating, a biocompatible coating, a non-biologic coating, or combinations thereof.

11. The augmented medical implant of claim 9, wherein the drug release mechanism configured to deliver cannabidiol at the site of implantation comprises a microfluidic device selected from: an actively driven reservoir, a passive micropump, an electrostatic micropump, an active micropump.

12. The augmented medical implant of claim 1, wherein the drug release mechanism configured to deliver cannabidiol at the site of implantation comprises an embedded fluid port to permit the drug release mechanism to refilled with cannabidiol.

13. The augmented medical implant of claim 1, wherein the drug release mechanism configured to deliver cannabidiol at the site of implantation comprises a reservoir associated with a controller, wherein the controller is operative to actuate release of cannabidiol from the reservoir.

14. The augmented medical implant of claim 13, wherein the controller is configured to be operative to actuate release of cannabidiol from the reservoir based upon a predetermined timing.

15. The augmented medical implant of claim 13, wherein the controller is configured to be operative to actuate release of cannabidiol from the reservoir based upon receipt of a release command.

16. The augmented medical implant of claim 13, wherein the controller is configured to be operative to actuate release of cannabidiol from the reservoir based upon detection of a physiological condition.

17. The augmented medical implant of claim 13, wherein the controller comprises a microchip.

18. The augmented medical implant of claim 9, wherein the wherein the drug release mechanism configured to deliver cannabidiol at the site of implantation comprises a depot selected from: a polymer formulation, a liposomal formulation, a micro-encapsulation formulation, an ion-exchange resin formulation, or combinations thereof.

19. The augmented medical implant of claim 1, wherein the drug release mechanism configured to deliver cannabidiol at the site of implantation comprises an adherent coating configured to elute cannabidiol, and wherein the physical association with the implantable medical device comprises the adherent coating being adhered to at least a portion of a surface of the implantable medical device.

20. The augmented medical implant of claim 19, wherein the implantable medical device comprises one or more of a stent, a tubing, a drain, an intra-ocular lens, a microneedle, a microcapsule, a microresevoir, a micropump, or combinations thereof.
